(19) United States
(12) Patent Application Publication
(10) Pub. No.: US 2015/0344613 A1
(43) Pub. Date: Dec. 3, 2015

(54) POLYTHIOUREA COMPOSITIONS AND METHODS OF USE

(71) Applicant: EMPIRE TECHNOLOGY DEVELOPMENT LLC, Wilmington, DE (US)

(72) Inventors: William Brenden CARLSON, Seattle, WA (US); Gregory D. PHELAN, Cortland, NY (US)

(73) Assignee: EMPIRE TECHNOLOGY DEVELOPMENT LLC, Wilmington, DE (US)

(21) Appl. No.: 14/291,124

(22) Filed: May 30, 2014

(51) Int. Cl. C08G 18/76; C07C 331/30; C08G 18/18; C08G 18/08; C09D 175/02; C08G 18/38

(52) U.S. Cl. CPC C08G 18/7664; C09D 175/02; C08G 18/3819; C08G 18/1816; C08G 18/14; C07C 331/30

(57) ABSTRACT

Disclosed herein are compositions and methods of making polythiourea and polythiourethane foams and coatings. In one embodiment, a method of preparing a polythiourea composition involves combining at least one polyamine compound, at least one polyisothiocyanate compound, and at least one catalyst to form a reaction mixture, and mixing the reaction mixture to form the polythiourea composition. In some embodiments, the polyamine compound may have an amine functionality of two or more, and is present in an amount of about 14 wt % to about 40 wt % of the reaction mixture. In some embodiments, the polyisothiocyanate compound may have a functionality of two or more, and is present in an amount of about 60 wt % to about 86 wt % of the reaction mixture. In some embodiments, the catalyst may be present in an amount of about 0 wt % to about 5 wt % of the reaction mixture.

### BACKGROUND

The physical and mechanical properties of polyurethane foams make them useful for a wide variety of applications, including thermal and sound insulation, upholstery and bedding, and packaging materials. Urethane foams are formed from the combination of isocyanates and polyols. However, the isocyanate group is a very strong Lewis acid and reacts rapidly with many electron donor species. For example, alcohol-, water-, and nitrogen-containing species can react rapidly with isocyanates. In addition, isocyanates are toxic as they react rapidly with DNA. Thus, there is a need to develop less toxic foams and polymer systems to replace polyurethanes.

### SUMMARY

Disclosed herein are compositions and methods of making polythiourea and polythiourethane foams and coatings. In one embodiment, a method of preparing a polythiourea composition involves combining at least one polyamine compound, at least one polyisothiocyanate compound, and at least one catalyst to form a reaction mixture, and mixing the reaction mixture to form the polythiourea composition. In some embodiments, the polyamine compound may have an amine functionality of two or more, and is present in an amount of about 14 wt % to about 40 wt % of the reaction mixture. In some embodiments, the polyisothiocyanate compound may have a functionality of two or more, and is present in an amount of about 60 wt % to about 86 wt % of the reaction mixture. In some embodiments, the catalyst may be present in an amount of about 0 wt % to about 5 wt % of the reaction mixture.

In another embodiment, a method of making a polyisothiocyanate compound includes contacting a polyamine compound with carbon disulfide to form a thiocarbamate compound, and contacting the thiocarbamate compound with an oxidizing agent to form the polyisothiocyanate compound.

In a further embodiment, a polythiourea composition is produced by a method that involves combining at least one polyamine compound, at least one polyisothiocyanate compound, and at least one catalyst to form a reaction mixture, and mixing the reaction mixture to form the polythiourea composition. In some embodiments, the polyamine compound may have an amine functionality of two or more, and is present in an amount of about 14 wt % to about 40 wt % of the reaction mixture. In some embodiments, the polyisothiocyanate compound may have a functionality of two or more, and is present in an amount of about 60 wt % to about 86 wt % of the reaction mixture. In some embodiments, the catalyst may be present in an amount of about 0 wt % to about 5 wt % of the reaction mixture.

In an additional embodiment, an article of manufacture may include any polythiourea composition prepared by the methods described herein.

In a further embodiment, a method for preparing a chemical resistant coating composition involves contacting at least one polyamine component, at least one polyisothiocyanate component, and at least one catalyst to form a reaction mixture, and mixing the reaction mixture to form a polythiourea prepolymer binder. In some embodiments, the polyamine compound may have an amine functionality of two or more, and is present in an amount of about 14 wt % to about 40 wt % of the reaction mixture. In some embodiments, the polyisothiocyanate compound may have a functionality of two or more, and is present in an amount of about 60 wt % to about 86 wt % of the reaction mixture. In some embodiments, the catalyst may be present in an amount of about 0 wt % to about 5 wt % of the reaction mixture.

In yet another embodiment, a method of neutralizing a chemical toxant involves contacting the chemical toxant with a polythiourea composition. The polythiourea composition is prepared by combining at least one polyamine compound, at least one polyisothiocyanate compound, and at least one catalyst to form a reaction mixture, and mixing the reaction mixture to form the polythiourea composition. In some embodiments, the polyamine compound may have an amine functionality of two or more, and is present in an amount of about 14 wt % to about 40 wt % of the reaction mixture. In some embodiments, the polyisothiocyanate compound may have a functionality of two or more, and is present in an amount of about 60 wt % to about 86 wt % of the reaction mixture. In some embodiments, the catalyst may be present in an amount of about 0 wt % to about 5 wt % of the reaction mixture.

In one embodiment, a method of preparing a polythiourethane composition involves combining at least one polyol, at least one polyisothiocyanate compound, and at least one catalyst to form a reaction mixture, and mixing the reaction mixture to form the polythiourethane composition. In some embodiments, the polyol may have a functionality of two or more, and is present in an amount of about 14 wt % to about 40 wt % of the reaction mixture. In some embodiments, the polyisothiocyanate compound may have a functionality of two or more, and is present in an amount of about 60 wt % to about 86 wt % of the reaction mixture. In some embodiments, the catalyst may be present in an amount of about 0 wt % to about 5 wt % of the reaction mixture.

In a further embodiment, a polythiourea composition includes about 14 weight percent to about 40 weight percent of units derived from a polyamine monomer having a functionality of two or more, and about 60 weight percent to about 86 weight percent of units derived from a polyisothiocyanate monomer having a functionality of two or more.

### BRIEF DESCRIPTION OF THE FIGURES

FIG. 1 depicts an exemplary mechanism of a polythiourea coating reacting with a chemical toxant according to an embodiment.

FIG. 2 depicts a structure of polythiourea chain stacked upon one another according to an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope.

“Unit derived from a monomer” denotes a unit which can be obtained directly from the monomer by polymerization. For example, units derived from a polyamine monomer denotes to the final polyamine units incorporated in the polymer after polymerization process. Thus, the terminology “unit derived from a monomer” relates only to the final composition of the polymer and is independent of the polymerization process used to synthesize the polymer.

Disclosed herein are compositions and methods of preparing polythiourea from polyamines and polyisothiocyanates. In some embodiments, a method of preparing a polythiourea composition involves combining at least one polyamine compound, at least one polyisothiocyanate compound, and at least one catalyst to form a reaction mixture, and mixing the reaction mixture to form the polythiourea composition. In some embodiments, the polyamine compound may have an amine functionality of two or more, and is present in an amount of about 14 wt % to about 40 wt % of the reaction mixture. In some embodiments, the polyisothiocyanate compound may have a functionality of two or more, and is present in an amount of about 60 wt % to about 86 wt % of the reaction mixture. In some embodiments, the catalyst may be present in an amount of about 0 wt % to about 5 wt % of the reaction mixture.

The overall reaction between the polyamine compound and the polyisothiocyanate compound can be represented as follows:

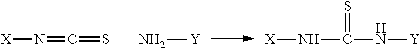

### Polyamine Compound

Non-limiting examples of the polyamine compound that may be used in making the polythiourea compound include a primary amine, a secondary amine, a tertiary amine, or any combination thereof. For example, the polyamine compound may be an aromatic polyamine, an aliphatic polyamine, a polyamino polyether, a polyamino polyester, a polyamino polycarbonate, a polyamino polycaprolactone, a polyamino acrylic, a polyamino styrenic, a polyamino polyamide, or any combination thereof. In some embodiments, the polyamine compound may be an aromatic diamine, a C₅-C₂₅ alkyl diamine, a C₅-C₂₅ alkenyl diamine, a C₅-C₂₅ alkynyl diamine, or any combination thereof.

In some embodiments, the polyamine compound may be amine-terminated hydrocarbons and may be represented as follows:

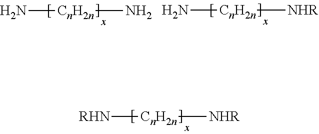

wherein n is an integer from 1 to 20; each R is independently, alkyl or aryl; and x is an integer from 1 to 20.

In some embodiments, the polyamine compound may be amine-terminated polyethers, and may be represented as follows:

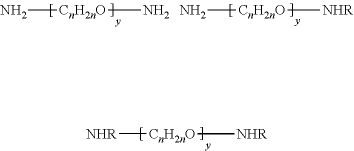

wherein n is an integer from 1 to 20; each R is independently, alkyl or aryl; and y is an integer from 1 to 20.

In some embodiments, the polyamine compound may be amine-terminated polyesters and can be represented as follows:

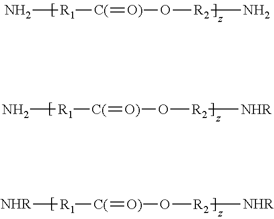

wherein z is an integer from 1 to 20; each R is independently, alkyl or aryl; each R₁ is independently alkylene group of 1 to 20 C atoms or arylene group; and each R₂ is independently alkylene group of 1 to 20 C atoms or arylene group.

Suitable aromatic diamines that may be used include, but are not limited to, 1,3-bis(aminomethyl)benzene, 1,4bis(aminomethyl)benzene, isophthalic acid dihydrazide, and 2,4-diaminotoluene, or any combination thereof. Examples of alicyclic diamines include, but are not limited to, 1,3-bis(aminomethyl)cyclohexane, 1,4 bis(aminomethyl)-cyclohexane, 3-aminomethyl-3,5,5-trimethylhexylamine, 1,2-diaminecyclohexane, 1,4-diaminocyclohexane, methylenebis(4-cyclohexylamine), piperazine, 2-piperazine, 2,5-dimethylpiperazine, 2,6-dimethylpiperazine, N,N′-bis(3-aminopropyl)piperazine, 1,3-di(4-piperidyl)propane, hydantoin, and hexahydro-1H-1,4-diazepine, or any combination thereof. Examples of aliphatic diamines include, but are not limited to, 1,6-diaminohexane, 1,7-diaminoheptane, 1,8-diaminooctane, 1,9-diaminononane, 1,10-diaminodecane, 1,12-diaminododecane, bis(2-aminoethyl)amine, bis(3-aminopropyl)amine, N,N′-bis(aminopropyl)methylamine, N-(3-aminopropyl)-1,4-butanediamine, N,N′-(3-aminopropyl)-1,4-butanediamine, adipic acid dihydrazide, dodecandioic acid, dihydrazide, sebacic acid dihydrazide, methylene bis 4-aminocyclohexane, and tetramethylene pentamine, or any combination thereof.

The polyamine compound disclosed herein may have an amine functionality of at least two, at least three, at least four, at least five, at least six, at least seven, or at least eight. In some embodiments, the polyamine compound may be present in the reaction mixture from about 14 wt % to about 40 wt %, from about 14 wt % to about 30 wt %, from about 14 wt % to about 25 wt %, or from about 14 wt % to about 20 wt %. Specific examples include, but are not limited to, about 14 wt %, about 20 wt %, about 25 wt %, about 30 wt %, about 35 wt %, and about 40 wt %, or any range between any two of these values (including the endpoints).

### Polyisothiocyanate Compound

The polyisothiocyanate compounds may be obtained commercially or may be synthesized from amines. An exemplary reaction mechanism is shown below:

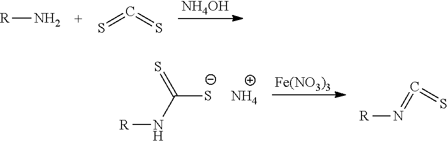

In some embodiments, a method of making a polyisothiocyanate compound includes contacting a polyamine compound with carbon disulfide to form a thiocarbamate compound, and contacting the thiocarbamate compound with an oxidizing agent to form the polyisothiocyanate compound.

The polyamine compound may be any polyamine compound described herein. In some embodiments, the polyamine is contacted with carbon disulfide in the presence of a base. Suitable bases that may be used include, but are not limited to, ammonium hydroxide and triethylamine. The nitrogen of the amine group in the polyamine compound is a Lewis base and reacts with the carbon of the carbon disulfide which is a Lewis acid. The reaction between the amine and carbon disulfide yields a thiocarbamate compound. The polyamine compound, carbon disulfide, and the base may be heated to a temperature of about 0° C. to about 30° C., about 0° C. to about 20° C., about 0° C. to about 15° C., or about 0° C. to about 10° C. Specific temperatures include, but are not limited to, about 0° C., about 5° C., about 10° C., about 15° C. about 20° C., and about 30° C., or any range between any two of these values (including the endpoints). The heating may be performed for about 30 minutes to about 6 hours, about 30 minutes to about 4 hours, about 30 minutes to about 2 hours, or about 30 minutes to about 1 hour. Specific examples include, but are not limited to, about 30 minutes, about 1 hour, about 3 hours, about 4 hours, and about 6 hours, or any range between any two of these values (including the endpoints).

The thiocarbamate compound formed is subsequently reacted with an oxidizing agent, such as a metal nitrate or tosyl chloride. The metal nitrate may be nitrate of any transition metal, such as iron, tin, lead, and the like. The thiocarbamate compound and the oxidizing agent may be heated to a temperature of about 0° C. to about 30° C., about 0° C. to about 20° C., about 0° C. to about 15° C., or about 0° C. to about 10° C. Specific temperatures include, but are not limited to, about 0° C., about 5° C., about 10° C., about 15° C. about 20° C., and about 30° C., or any range between any two of these values (including the endpoints). The polyisothiocyanate compound formed by the methods described herein may be purified by subsequent heating and cooling steps, using techniques known in the art.

In some embodiments, the polyisothiocyanate compound may be an alkyl diisothiocyanate, an alkyl triisothiocyanate, an alkenyl diisothiocyanate, an alkenyl triisothiocyanate, an aryl diisothiocyanate, an aryl triisothiocyanate, or any combination thereof. For example, the polyisothiocyanate compound includes a C₅-C₂₅ alkyl diisothiocyanate, a C₅-C₂₅ alkenyl diisothiocyanate, a C₅-C₂₅ alkynyl diisothiocyanate, or any combination thereof. Other non-limiting examples include, but are not limited to, triphenylmethane-4,4′4″-triisothiocyanate, benzene-1,3,5-triisothiocyanate, toluene-2,4,6-triis othiocyanate, hexamethylene diisothiocyanate, xylylene diisothiocyanate, chlorophenylene diisothiocyanate, diphenylmethane-4,4′-diisothiocyanate, naphthalene-1,5-diisothiocyanate, 3,3′-dimethyl-4,4′biphenylene diisthioocyanate, 3-3′dimethoxy-4-biphenylene diisothiocyanate, 2′,3,3′-dimethyl-4,4′-biphenylene diisothiocynate, 5,5′-tetramethyl-4-biphenylene diisothiocyanate, 2,2′,5,5′-tetramethyl-4,4′biphenylene diisothiocyanate, 4-methylenebis(phenylisothiocyanate), 4,4′-sulfonylbis(phenylisothiocyanate), 4,4′-methylene di-orthototylisothiocyanate, ethylene diisothiocyanate, ethylene diisothiocyanate, trimethylenediisothiocyanate, diphenyl diisothiocyanate, 1,3-bis(isocyanatemethyl)benzene, 1,3-bis(1-isocyanate-1-methylethyl)benzene, methylene bis(phenylisothiocyanate), methylene bis 4-isothiocyantocyclohexane, hexamethylene diisothiocyanate, and 2,4-toluene diisothiocyanate, or any combination thereof.

Suitable aromatic alkyl isothiocyanates that may be used include, but are not limited to, 1,3-bis(isothiocyanatemethyl)benzene, 1,3-bis(1-isocyanate-1-methylethyl)benzene, and the like. Examples of alicyclic isothiocyanates 1,3-bis(isothiocyanatemethyl)cyclohexane, 3-isothiocyanatemethyl-3,5,5-trimethylhexylisothiocyanate, methylenebis(4-cyclohexylisothiocyanate), and 2,5(2,6)-bis(isothiocyanatemethyl)bicycle[2,2,1]heptane. Further, other aliphatic polyisothiocyanates that may be used include, but are not limited to, 1,6-diisothiocyanate hexane, 1,5-diisothiocyanate-2-methyl pentane, 1,8-diisothiocyanate octane, 1,12-diisothiocyanate dodecane, tetraisothiocyanate silane, and monomethyltriisothiocyanate silane.

The polyisothiocyanate compound disclosed herein may have a functionality of at least two, at least three, at least four, at least five, at least six, at least seven, or at least eight. In some embodiments, the polyisothiocyanate compound may be present in the reaction mixture from about 60 wt % to about 86 wt %, about 60 wt % to about 80 wt %, about 60 wt % to about 70 wt %, or about 60 wt % to about 65 wt %. Specific examples include, but are not limited to, about 60 wt %, about 65 wt %, about 70 wt %, about 75 wt %, about 80 wt %, and about 86 wt %, or any range between any two of these values (including the endpoints).

Any polyamine compound disclosed herein may be combined with any of the polyisothiocyanate compound. For example, in one embodiment, the polyamine compound is 2-{2-[2-(2-amino-ethoxy)-ethoxy]-ethoxy}-ethylamine and the polyisothiocyanate compound is 4,4′-methylene diphenyl diisothiocyanate. In another embodiment, the polyamine component is octadeca-1,18 diamine and the polyisothiocyanate component is octadeca-1,18 diisothiocyanate.

### Catalysts

The reaction between the isothiocyanate and the amine group in the polyamine compound may take place without a catalyst, but at a slow rate, especially at colder temperatures. Without a catalyst, a foam may expand, but it may not cure adequately to give good physical properties. Therefore, suitable catalysts may be used in making a polythiourea composition, such as a bismuth catalyst, a zinc catalyst, a tin catalyst, and an amine, or any combination thereof. Some non-limiting examples of catalysts include, but are not limited to, zinc octoate, stannous octoate, bis-butyltin dilaurate, bis-butyltin diacetate, bis-butyltin dimethoxide, dimethyl-bis[1-oxonedecyl)oxy]stannane, di-n-octyltin bis-isooctyl mercaptoacetate, triethylenediamine, triethylamine, 1,3,5-cycclohexyltriamine, tributylamine, N-ethylmorpholine, N-methyl-morpholine, N-methylpiperazine, trimethylene diazine, tin (II) chloride, and tin (IV) chloride, or any combination thereof.

In some embodiments, the catalyst may be present in the reaction mixture from about 0 wt % to about 5 wt %, from about 0.1 wt % to about 5 wt %, from about 1 wt % to about 5 wt %, or from about 2 wt % to about 5 wt %. Specific examples include, but are not limited to, about 0 wt %, about 0.1 wt %, about 0.5 wt %, about 1 wt %, about 2 wt %, and about 5 wt %, or any range between any two of these values (including the endpoints).

### Curing Agent

In some embodiments, the reaction mixture may also include one or more curing agents, such as, for example, trimethylamine, triethylamine, N-methylmorpholine, N-ethylmorpholine, N,N-dimethylbenzylamine, N,N-dimethylethanolamine, N,N,N′,N′-tetramethyl-1,4-butanediamine, N,N-dimethylpiperazine, 1,4-diazobicyclo-2,2,2-octane, bis(dimethylaminoethyl)ether, bis(2-dimethylaminoethyl) ether, morpholine, 4,4′-(oxydi-2,1-ethanediyl)bis, triethylenediamine, pentamethyl diethylene triamine, dimethyl cyclohexyl amine, N-cetyl N,N-dimethyl amine, N-coco-morpholine, N,N-dimethyl aminomethyl N-methyl ethanol amine, N,N,N′-trimethyl-N′-hydroxyethyl bis(aminoethyl) ether, N,N-bis(3-dimethylaminopropyl)N-isopropanolamine, (N,N-dimethyl)amino-ethoxy ethanol, N,N,N′,N′-tetramethyl hexane diamine, 1,8-diaza-bicyclo-5,4,0-undecene-7, N,N-dimorpholinodiethyl ether, N-methyl imidazole, dimethyl aminopropyl dipropanolamine, bis(dimethylaminopropyl)amino-2-propanol, tetramethylamino bis(propylamine), (dimethyl(aminoethoxyethyl))((dimethyl amine)ethyl)ether, tris(dimethyl-aminopropyl)amine, dicyclohexyl methyl amine, bis(N,N-dimethyl-3-aminopropyl)amine, 1,2-ethylene piperidine, and methyl-hydroxyethyl piperazine, or any combination thereof.

### Additives

Additional optional materials included in a reaction mixture for making polythiourea include, but are not limited to, wetting agents, surfactants, accelerators, activators, antioxidants, fillers, coloring agents, inhibitors, and nucleating agents, or any combination thereof. For example, wetting additives may be added to the modified curative blends of the polythiourea composition to more effectively disperse the pigment(s). Accelerators (or promoters) may be used in small proportions to increase the reaction rate of the mixture. Activators may also be included to increase the effectiveness of an accelerator, and may speed up the reaction in unison with a catalyst. Further, antioxidants may be used to retard deterioration caused by oxidation. Those of ordinary skill in the art are aware of the purpose of these additives and the amounts that should be employed to fulfill those purposes.

### Reaction Conditions

In some embodiments, the reaction mixture is mixed and heated to a temperature of about 20° C. to about 70° C., about 20° C. to about 60° C., about 20° C. to about 50° C., or about 20° C. to about 40° C. Specific temperatures include, but are not limited to, about 20° C., about 30° C., about 40° C., about 50° C. about 60° C., and about 70° C., or any range between any two of these values (including the endpoints). The heating may be performed for about 5 minutes to about 12 hours, about 30 minutes to about 12 hours, about 2 hours to about 12 hours, or about 6 hours to about 12 hours. Specific examples include, but are not limited to, about 5 minutes, about 30 minutes, about 1 hour, about 3 hours, about 6 hours, and about 12 hours, or any range between any two of these values (including the endpoints).

The polythiourea compositions formed by the methods described herein may have an NCS content from about 2 wt % to about 80 wt %, from about 2 wt % to about 60 wt %, from about 2 wt % to about 40 wt %, or from about 2 wt % to about 20 wt % of the total polymer weight. Specific examples include, but are not limited to, about 2 wt %, about 10 wt %, about 20 wt %, about 30 wt %, about 50 wt %, about 60 wt %, and about 80 wt %, or any range between any two of these values (including the endpoints).

In some embodiments, the polythiourea compositions may be modified so that they include additional function groups, such as acrylic functional groups, vinyl functional groups, epoxy functional groups, or any combination thereof. The polythiourea compositions may be tailored to form a foam, a film, or a coating, or any other article, depending on the required needs.

The polythiourea compositions disclosed herein may have units derived from polyamine monomers from about 14 wt % to about 40 wt %, from about 14 wt % to about 30 wt %, from about 14 wt % to about 25 wt %, or from about 14 wt % to about 20 wt % of the total polymer weight. Specific examples include, but are not limited to, about 14 wt %, about 20 wt %, about 25 wt %, about 30 wt %, about 35 wt %, and about 40 wt %, or any range between any two of these values (including the endpoints). In some embodiments, the polythiourea compositions disclosed herein may have units derived from polyisothiocyanate monomers from about 60 wt % to about 86 wt %, about 60 wt % to about 80 wt %, about 60 wt % to about 70 wt %, or about 60 wt % to about 65 wt % of the total polymer weight. Specific examples include, but are not limited to, about 60 wt %, about 65 wt %, about 70 wt %, about 75 wt %, about 80 wt %, and about 86 wt %, or any range between any two of these values (including the endpoints).

### Polythiourethane Compositions

Also disclosed herein are compositions and methods for making polythiourethanes. In some embodiments, a method of preparing polythiourethane composition includes combining at least one polyol, at least one polyisothiocyanate compound, and at least one catalyst to form a reaction mixture, and mixing the reaction mixture to form the polythiourethane composition. In some embodiments, the polyol may have a functionality of two or more, and is present in an amount of about 14 wt % to about 40 wt % of the reaction mixture. In some embodiments, the polyisothiocyanate compound may have a functionality of two or more, and is present in an amount of about 60 wt % to about 86 wt % of the reaction mixture. In some embodiments, the catalyst is present in an amount of about 0 wt % to about 5 wt % of the reaction mixture.

The polyisothiocyanate compound used in the reaction mixture may be any polyisothiocyanate compound that is described herein. Non-limiting examples of polyols include, but are not limited to, pentaerythritol, sorbitol, sucrose, glucose, fructose, a polyether, and a polyester, or any combination thereof. In addition, suitable diols that may be used include, but are not limited to, ethylene glycol, 1,4-butanediol, 1,5-pentanediol, 1,6-hexanediol, 1,10-decanediol, 2,2-dimethyl-1,3-propanediol, 1,3 propanediol, trimethylpropanol, bisphenol, diethylene glycol, dipropylene glycol, and 1,1,3-trimethyltriethylene diol. Polyalkadiene polyols prepared from dienes, such as 1,3-butadiene, isoprene, chloroprene, 2-cyano-1,3-butadiene, 2,3-dimethyl-1,2-butadiene, may also be used.

Suitable catalysts may be used in making a polythiourethane composition are a bismuth catalyst, a zinc catalyst, a tin catalyst, an amine, or any combination thereof. Some non-limiting examples of catalysts include, but are not limited to, zinc octoate, stannous octoate, bis-butyltin dilaurate, bis-butyltin diacetate, bis-butyltin dimethoxide, dimethyl-bis[1-oxonedecyl)oxy]stannane, di-n-octyltin bis-isooctyl mercaptoacetate, triethylenediamine, triethylamine, 1,3,5-cycclohexyltriamine, tributylamine, N-ethylmorpholine, N-methyl-morpholine, N-methylpiperazine, trimethylene diazine, tin (II) chloride, and tin (IV) chloride, or any combination thereof. Additional materials included in a reaction mixture for making polythiourea include, but are not limited to, wetting agents, surfactants, accelerators, activators, antioxidants, fillers, coloring agents, inhibitors, nucleating agents, or any combination thereof.

In some embodiments, the reaction mixture comprising at least one polyol, at least one polyisothiocyanate compound, and at least one catalyst is mixed and heated to a temperature of about 100° C. to about 250° C., about 100° C. to about 200° C., about 100° C. to about 150° C., or about 100° C. to about 110° C. Specific temperatures include, but are not limited to, about 100° C., about 130° C., about 150° C., about 180° C. about 200° C., and about 250° C., or any range between any two of these values (including the endpoints). The heating may be performed for about 5 minutes to about 12 hours, about 30 minutes to about 12 hours, about 2 hours to about 12 hours, or about 6 hours to about 12 hours. Specific examples include, but are not limited to, about 5 minutes, about 30 minutes, about 1 hour, about 3 hours, about 6 hours, and about 12 hours, or any range between any two of these values (including the endpoints).

### Foams

In some embodiments, the polythiourea and polythiourethane compositions disclosed herein may be prepared into foams by adding a blowing agent in the reaction mixture. Non-limiting examples of blowing agents include, but are not limited to, carbon dioxide, nitrogen, argon, azo compounds, azide salts, methylene chloride, chlorofluorocarbons, hydrazine, pentane, and isopentane, or any combination thereof. By adjusting the amount of the blowing agent, the density of the foam may be varied, for example, from about 10 to about 250 kilograms per cubic meter, from about 10 to about 200 kilograms per cubic meter, from about 10 to about 150 kilograms per cubic meter, or from about 10 to about 100 kilograms per cubic meter. Specific examples include, but are not limited to, about 10 kilograms per cubic meter, about 50 kilograms per cubic meter, about 100 kilograms per cubic meter, about 150 kilograms per cubic meter, about 250 kilograms per cubic meter, or any range between any two of these values (including the endpoints).

Surfactants may also be included in the reaction process which may decrease the bubble size in the foam. Surfactants may be used to modify the characteristics of the polymer during the foaming process. They may be used to emulsify the liquid components, regulate cell size, and/or stabilize the cell structure to prevent collapse and surface defects. Rigid foam surfactants are designed to produce very fine cells and a very high closed cell content. Flexible foam surfactants are designed to stabilize the reaction mass while at the same time maximize open cell content to prevent the foam from shrinking. The type of the surfactant may depend on choice of isothiocyanate, polyamine, component compatibility, system reactivity, process conditions and equipment, tooling, part shape, and shot weight.

In some embodiments, the steps of making polythiourea and polythiourethane foams may involve the following sequential steps: 1) bubble generation and growth; 2) packing of the bubble network and cell window stabilization; 3) polymer stiffening and cell opening; and 4) final curing.

During bubble generation, air bubbles may be introduced in the mixing step using a high speed mixer. These bubbles may serve as starting points for the foam growth. The energy required for formation and growing of a bubble is related to the surface tension of the surfactant solution. Silicone surfactants can reduce surface tension and, thus, reduce the energy needed to form a bubble. As a result, the number of bubbles introduced into the system as well as the final foam cell size may be determined by the surface tension lowering ability of the added surfactant.

During packing of the bubble network, bubbles may enlarge and touch each other and squeeze out most of the material between them. The maximum foam expansion may occur at this stage of the process. During polymer stiffening and cell opening, windows where one bubble touches another bubble may pop, leaving open cells that are connected together by struts of polythiourea. If the bubbles are small enough, the struts may support the weight of the foam, and prevent the foam from collapse. The open cells may give the foam its ability to absorb water. In a closed cell foam, each individual cell is more or less spherical in shape and completely closed in by a wall of plastic. In an open cell foam, individual cells are inter-connecting, as in a sponge. A closed cell foam makes a better buoy or life jacket because the cells do not fill with liquid. For pigging applications, it is desirable to have a material that can soak up water and other contaminants so they can be removed and the pipeline can be dried. Adding too much surfactant can turn an open cell foam into a closed cell foam.

The polythiourea and polythiourethane foams of the disclosure make it possible to produce articles exhibiting suitable properties, such as high the tensile strength, and the tear strength. Other properties that are also important for certain applications are abrasion resistance, resistance to shrinkage during molding, compression, and resilience.

Fillers may be added during the process of making polythiourea and polythiourethane foams to affect rheological and mixing properties, specific gravity (density-modifying fillers), the modulus, the tear strength, reinforcement, and the like. The fillers are generally inorganic, and suitable fillers include, but are not limited to, numerous metals, metal oxides and salts, such as zinc oxide and tin oxide, as well as barium sulfate, zinc sulfate, calcium carbonate, zinc carbonate, barium carbonate, clay, tungsten, tungsten carbide, an array of silicas, regrind (recycled core material typically ground to 30 mesh particle), glass spheres (solid or hollow, and filled or unfilled), fibers, and mixtures thereof. The selection of such filler(s) is dependent upon the type of article desired.

### Paints and Coatings

In some embodiments, the polythiourea and polythiourethane compositions described herein may be part of paint and coating compositions. The coating compositions may be a latex emulsion, non-aqueous dispersion, or powder. In some embodiments, a method for preparing a chemical resistant coating composition may involve contacting at least one polyamine component, at least one polyisothiocyanate component, and at least one catalyst to form a reaction mixture, and mixing the reaction mixture to form a polythiourea prepolymer binder. In some embodiments, the polyamine compound may have an amine functionality of two or more, and is present in an amount of about 14 wt % to about 40 wt % of the reaction mixture. In some embodiments, the polyisothiocyanate compound may have a functionality of two or more, and is present in an amount of about 60 wt % to about 86 wt % of the reaction mixture. In some embodiments, the catalyst may be present in an amount of about 0 wt % to about 5 wt % of the reaction mixture. Similar procedures may be used to react polyols and polysiothiocyanates to form polythiourethane prepolymers.

The polythiourea and polythiourethane prepolymers disclosed herein may be linear or branched in nature, and range from several hundred in molecular weight to a few thousand. In some embodiments, an isothiocyanate terminated prepolymer may be formed by using isothiocyanate compound in excess in the reaction process. Similarly, an amine terminated prepolymer may be formed by using polyamine compound in excess. An exemplary reaction is shown below:

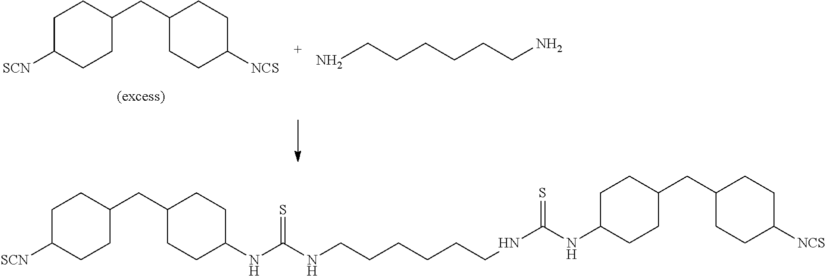

For example, one or more polythiourea or polythiourethane prepolymer described herein may be linked to each other to form a binder. In some embodiments, the polythiourea prepolymer may be crosslinked to an acrylic binder, a vinyl binder, or a styryl binder, or any combination thereof.

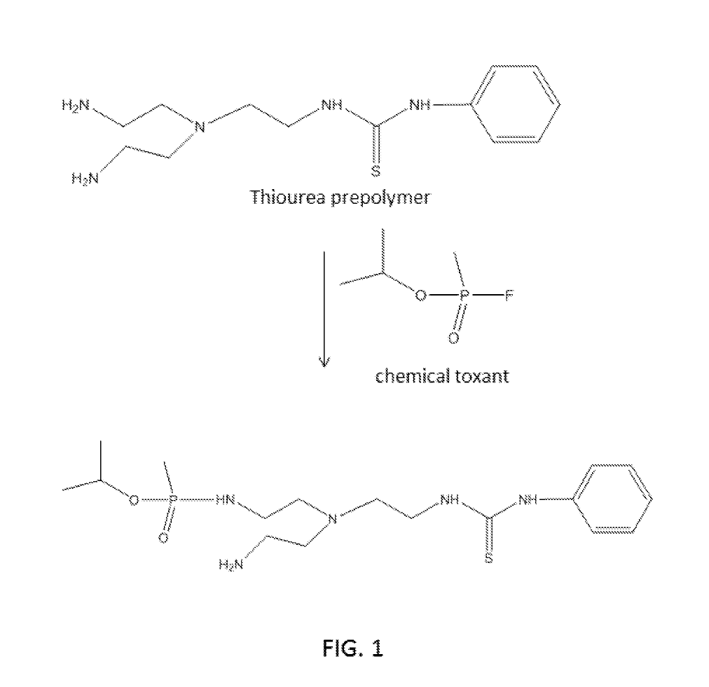

In some embodiments, the polythiourea or polythiourethane prepolymer may include at least one amine functional group that is capable of neutralizing a chemical toxant. In other embodiments, polythiourea prepolymer may be modified to include at least one oxidizing agent that is capable of neutralizing a chemical toxant. Non-limiting examples of such oxidizing agents include, but are not limited to, a hydantoin, a nitro group, a nitroso group, a triazine, an oxazolidinone, an imidazolidinone, and an N-halamine, or any combination thereof. For example, the nitrogen moieties in polythiourea may react with the fluoride and/or chloride groups of the chemical toxants and neutralize them. An exemplary mechanism is shown in FIG. 1. For example, the oxidizing agent or the amine functional group of the polythiourea may oxidize the chemical toxant. In other embodiments, the oxidizing agent or the amine functional group may neutralize the chemical toxant through nucleophilic attack. Many of the chemical toxants have halo groups. When they come into contact with amine groups of the polythiourea prepolymer, a SN2 reaction may take place which destroys and locks down the toxic agent.

The chemical toxants include, but are not limited to, O-alkyl phosphono-fluoridates, such as sarin and soman, o-alkyl phophoramidocyanidates, such as tabun, O-alkyl, S-2-dialkyl aminoethyl alkylphosphonothiolates and corresponding alkylated or protonated salts, such as VX, mustard compounds, including 2-chloroethylchloromethylsulfide, bis(2-chloroethyl)sulfide, bis(2-chloroethylthio)methane, 1,2-bis(2-chloroethylthio)ethane, 1,3-bis(2-chloroethylthio)-n-propane, 1,4-bis(2-chloroethylthio)-n-butane, 1,5-bis(2-chloroethylthio)-n-pentane, bis(2-chloroethylthiomethyl)ether, and bis(2-chloroethylthioethyl)ether, Lewisites, including 2-chlorovinyldichloroarsine, bis(2-chlorovinyl)chloroarsine, tris(2-chlorovinyl)arsine, bis(2-chloroethyl)ethylamine, and bis(2-chloroethyl)methylamine, saxitoxin, ricin, alkyl phosphonyldifluoride, alkyl phosphonites, chlorosarin, chlorosoman, amiton, 1,1,3,3,3,-pentafluoro-2-(trifluoromethyl)-1-propene, 3-quinuclidinyl benzilate, methylphosphonyl dichloride, dimethyl methylphosphonate, dialkyl phosphoramidic dihalides, dialkyl phosphoramidates, arsenic trichloride, diphenyl hydroxyacetic acid, quinuclidin-3-ol, dialkyl aminoethyl-2-chlorides, dialkyl aminoethan-2-ols, dialkyl aminoethane-2-thiols, thiodiglycols, pinacolyl alcohols, phosgene, cyanogen chloride, hydrogen cyanide, chloropicrin, phosphorous oxychloride, phosphorous trichloride, phosphorus pentachloride, alkyl phosphites, sulfur monochloride, sulfur dichloride, and thionyl chloride. other chemical toxants include tris(2-chloroethyl)amine, 2-chloroethylchloro-methylsulfide, bis(2-chloroethyl) sulfide, bis(2-chloroethylthio) methane, bis(2-chloroethylthiomethyl) ether, ethyldichloroarsine, methyl-dichloroarsine, phenyldichloroarsine, phosgene oxime, chloropicrin, diphosgene, cyclosarin, 2-(dimethylamino)ethyl N,N-dimethylphosphoramidofluoridate, Novichok agents, s-[2-(diethylamino)ethyl]o-ethyl ethylphosphonothioate, ethyl ({2-[bis(propan-2-yl)amino]ethyl}sulfanyl)(methyl)phosphinate, O,O-Diethyl s-[2-(diethylamino)ethyl]phosphorothioate, and s-[2-(Diethylamino)ethyl]O-ethyl methylphosphonothioate, or any combination thereof.

In additional embodiments, the coating composition may further contain one or more additives. These additives may alter properties of the paint made from the coating composition, such as shelf life, application and longevity, and/or health and safety. Such additives may be added, for example, during the manufacture of emulsion polymers in the paint or during the formulation of the paint itself. Illustrative optional additives include, but are not limited to, initiators, rheology modifiers, preservatives, and the like. Initiators are a source of free radicals to initiate the polymerization process in which monomers form polymers. Coating compositions may contain a redox system initiator, such as ferrous and thiosulfate along with the persulfate salts, that promote polymerization at room temperature.

In some embodiments, thickeners and rheology modifiers may also be added to coating compositions to achieve desired viscosity and flow properties. Thickeners form multiple hydrogen bonds with the acrylic polymers, thereby causing chain entanglement, looping and/or swelling which results in volume restriction. Thickeners, such as cellulose derivatives including hydroxyethyl cellulose, methyl cellulose, and carboxymethyl cellulose, may be used in the coating compositions.

In some embodiments, one or more preservatives may be added in the coating compositions in low doses to protect against the growth of microorganisms. Preservatives, such as methyl benzisothiazolinones, chloromethylisothiazolinones, barium metaborate and 1-(3-chloroallyl)-3,5,7-triaza-1-azoniaadamantane chloride, may be used.

In some embodiments, the coating composition may further contain one or more of the following additives: solvents, pigments, plasticizers, surfactants and the like. Surfactants may be used, for example, to create the micelles for particle formation, as well as long-term particle stabilization. Surfactants may provide stability through electrostatic and steric hindrance mechanisms. Both ionic and non-ionic surfactants may be used. Examples may include, but are not limited to, alkyl phenol ethoxylates, sodium lauryl sulfate, dodecylbenzene sulfonate, polyoxyethylene alkyl ethers, polyoxyethylene alkyl allyl ethers, acetylene glycols, polyoxyethylene, stearic acid, and polyoxypropylene.

Coalescing agents, such as ester alcohols, benzoate ethers, glycol ethers, glycol ether esters and n-methyl-2-pyrrolidone, may be added to the coating compositions. Coalescing agents may be added to, for example, insure film formation under varying atmospheric conditions. They may be slow evaporating solvents with some solubility in the polymer phase. They may also act as a temporary plasticizer, allowing film formation at temperatures below the system's glass transition temperature. After film formation, the coalescing agents may slowly diffuse to the surface and evaporate, increasing the hardness and block resistance of the film.

In some embodiments, one or more plasticizers may be added to the compositions to adjust the tensile properties of the paint film. Plasticizers include, for example, a glucose-based derivative, a glycerine-based derivative, propylene glycol, ethylene glycol, phthalates and the like.

A paint, according to the disclosure, may further include one or more pigments. The term “pigments” is intended to embrace, without limitation, pigmentary compounds employed as colorants, including white pigments, as well as ingredients commonly known in the art as “opacifying agents” and “fillers”. Pigments may be any particulate organic or inorganic compound and may provide coatings the ability to obscure a background of contrasting color (hiding power). In some embodiments, the coating may further include photocatalytic pigments, such as titanium dioxide, zinc oxide, tin oxide, tungsten oxide, chromium oxide, hematite, magnetite, wiistite, or any combination thereof. The photocatalytic properties of these pigments may result from the promotion of electrons from the valence band to the conduction band under the influence of an electromagnetic spectrum. The reactive electron-hole pairs that are created migrate to the surface of these pigment particles where the holes oxidize adsorbed water to produce reactive hydroxyl radicals and the electrons reduce adsorbed oxygen to produce superoxide radicals, both of which can degrade organic compounds and grease sticking to the surface of a coating. In addition to self-cleaning properties, the photocatalytic pigments in paints may also provide hydrophilic properties to a coating.

### Films

In some embodiments, the polythiourea and polythiourethane compositions described herein may act as barrier films that can be used in a wide variety of consumer and industrial products. Such films may be produced by reacting C₅-C₂₅ linear diamines with alkyl diisothiocyanates. An exemplary reaction is presented below:

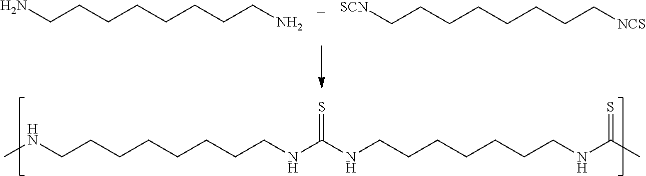

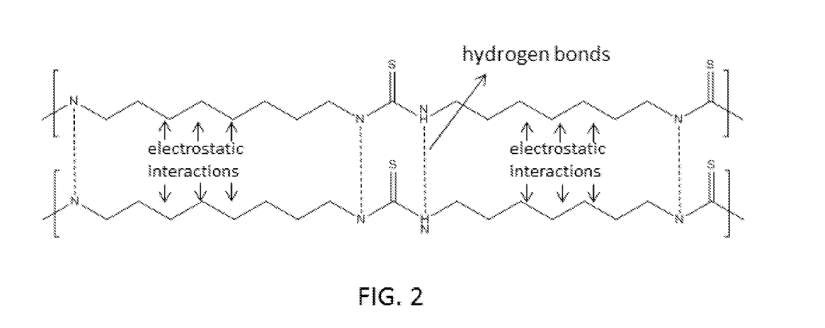

Since these polymeric chains are hydrophobic, they can stack upon each other into self-assembled structures. The electrostatic interactions between the two polymeric chains may contribute to the stability of the structure. Further, the thiourea linkages may form inter-chain hydrogen bonds and provide rigidity to the structures. An exemplary structure is shown in FIG. 2.

In some embodiments, the polythiourea and polythiourethane films described herein may have a thickness of about 0.1 micron to about 5 microns, about 0.1 micron to about 4 microns, about 0.1 micron to about 3 microns, about 0.1 micron to about 2 microns, about 0.1 micron to about 1 micron, or about 0.1 micron to about 0.5 microns. Specific examples include, but are not limited to, about 0.1 micron, about 0.5 micron, about 1 micron, about 2 microns, about 3 microns, about 4 microns, and about 5 microns, or any range between any two of these values (including the endpoints).

### Uses

The polythiourea and polythiourethane foams disclosed herein may be used as a packaging material, a protective film, a bag, a container, a wrapper, an automobile part, a sporting good, a tool, a cooking equipment, a heating equipment, and the like. Specifically, the foams disclosed herein may be incorporated as the absorptive material in a wound dressing and/or bandage. The foam may also be used to absorb bodily fluids, such as blood. Further, the films disclosed herein may have wide applications, such as being used in a semi-interpenetrating polymer network, aerospace industries, electronics displays, tablets, computers, cell phones, clothing, furniture, transparent films, and packaging materials.

The polythiourea and polythiourethane coatings described herein may be a decorative coating, an industrial coating, a protective coating, a UV-protective coating, a self-cleaning coating, a biocidal coating, or a chemical resistant coating, or any combination thereof. In some embodiments, the polythiourea coatings may be tailored for indoor or outdoor use. For example, aliphatic or cycloaliphatic polyamines may be used for outdoor coatings, and aromatic polyamines may be used for indoor coatings. The coatings may generally be applied to any substrate. The coated substrate may be an article, an object, a vehicle or a structure. Although no particular limitation is imposed on the substrate to be used in the present disclosure, exemplary substrates include an exterior of a building, vehicles, bridges, airplanes, metal railings, fences, glasses, plastics, ceramics, wood, stones, cement, fabric, paper, leather, walls, pipes, vessels, medical devices, kitchen appliances, kitchen cabinets, outdoor furniture, aluminum siding, metal products, washing machines, ships, chemical plants, automobiles, farm implements, containers, floor surfaces, and the like. The coating may be applied to the substrates described herein by any known technique in the art, such as spraying, dipping, rolling, brushing, or any combination thereof.

### EXAMPLES

### Example 1

### Preparation of a Polythiourea Foam

2-{2-[2-(2-amino-ethoxy)-ethoxy]-ethoxy}-ethylamine (97.5 parts), 1,3,5-cyclohexyltriamine (4 parts), 2-(2-ethoxy-ethoxy)-ethylamine (6 parts), dibutyltin dilaurate (0.04 part), hexafluoroethane (1.44 parts), foam stabilizer Tegostab B-8716LF (0.35 part) and foam stabilizer Tegostab B-8229 (0.35 part) are mixed to obtain a polyamine liquid, and adjusted to a temperature of 21±1° C.

About 162.7 parts of 4,4′-methylene diphenyl diisothiocyanate is added to the above obtained polyamine liquid and stirred for 5 minutes by a mixer (rotational speed: 1,425 rpm). The mixture is injected at room temperature into a wooden box of 300 mm×300 mm×300 mm with an open top and lined with a plastic sheet, to obtain a flexible slab foam. The obtained flexible polythiourea foam is taken out and left to cure for 24 hours. The polythiourea foam will exhibit high the tensile strength and the tear strength.

### Example 2

### Preparation of Isothiocyanate Terminated Polythiourea

About 1 liter of methylene chloride is mixed with 316.477 grams (1.074 mol, 2.149 mol NCS groups) of methylene bis(cyclohexene-4-isothiocyanate). A methylene chloride solution containing 100 grams (0.861 mol, 1.722 mol NH₂ groups) of hexamethylenediamine and 10 grams (0.0774 mol, 0.232 mol NH₂ groups) of 1,3,5-cyclohexanetriamine is added dropwise to the methylene chloride solution over a period of an hour. The mixture is stirred for 12 hours at room temperature under argon, and the methylene chloride is removed by rotary evaporation yielding a clear viscous liquid. The resulting product is a complex mixture of methylenebis(4-cyclohexylisothiocyanate) linear and branched oligomers, with a NCS equivalent weight of 580. The polythiourea foam will exhibit high the tensile strength and the tear strength.

### Example 3

### Preparation of an Amine Terminated Polythiourea

About 1 liter of methylene chloride is mixed with 265 grams (0.900 mol, 1.800 mol NCS groups) of methylene bis(cyclohexene-4-isothiocyanate). A methylene chloride solution containing 100 grams (0.861 mol, 1.722 mol NH₂ groups) of hexamethylenediamine and 10 grams (0.0774 mol, 0.232 mol NH₂ groups) of 1,3,5-cyclohexanetriamine is added dropwise to the methylene chloride solution over a period of an hour. The mixture is stirred for 12 hours at room temperature under argon, and the methylene chloride is removed by rotary evaporation yielding a clear viscous liquid. The resulting product is a complex mixture of methylenebis(4-cyclohexylisothiocyanate) linear and branched oligomers, with a NCS equivalent weight of 605. The polythiourea foam will exhibit high abrasion resistance.

### Example 4

### Preparation of a Polythiourea Coating

The coating is prepared as follows:

Component I: Chrome oxide green 44.21 grams, cobalt spinnel 30.92 grams, red iron oxide 0.77 grams, carbazole violet 2.25 grams, magnesium ferrite 9.68 grams, polymeric flattening agent 58.28 grams, deionized water 108.97 grams, polyethyleneimine (10 kDa MW) 189.88 grams, defoamer 1.18 grams, micromilled calcium carbonate 1.49 grams, anti-settling agent (6.0% solids) 74.121 grams, dispersing agent 2.24 grams, non-ionic emulsion (35% solids) 38.73 grams, and mercapiopropyltrimethoxysilane 3.67 grams.

Component II: Methylene bis(cyclohexene-4-isothiocyanate) 191.0 grams, and alkylacetate 34.77 grams.

Component III: Deionized water 141.53 grams.

A grind base is made of component I using a ball mill. The base is milled until a Hegman Grind of 6.5 or greater is reached. Component II is then blended into the mix using an impeller blade at 1000 rpm for 10 minutes and component III (de-ionized water) is added slowly (over a period of 15 minutes) to the mixture. The viscosity of the coating ranges from 100-300 centistrokes. The coating will exhibit self-cleaning and hydrophilic properties.

### Example 5

### Preparation of a Polythiourea Coating

The coating is prepared as follows:

Component I: Chrome oxide green 44.21 grams, cobalt spinnel 30.92 grams, red iron oxide 0.77 grams, carbazole violet 2.25 grams, magnesium ferrite 9.68 grams, polymeric flattening agent 58.28 grams, deionized water 108.97 grams, polythiourea of Example 3—199.23 grams, defoamer 1.18 grams, micromilled calcium carbonate 1.49 grams, anti-settling agent (6.0% solids) 74.121 grams, dispersing agent 2.24 grams, non-ionic emulsion (35% solids) 38.73 grams, and mercapiopropyltrimethoxysilane 3.67 grams.

Component II: Isothiocyanate terminated polythiourea of Example 2—191.0 grams, and alkylacetate 34.77 grams.

Component III: Deionized water 141.53 grams

A grind base is made of component I using a ball mill. The base is milled until a Hegman Grind of 6.5 or greater is reached. Component II is then blended into the mix using an impeller blade at 1000 rpm for 10 minutes and component III (de-ionized water) is added slowly (over a period of 15 minutes) to the mixture. The viscosity of the coating ranges from 100-300 centistrokes. The coating will act as a chemical resistant coating and oxidize chemical toxants.

### Example 6

### Preparation of a Diisothiocyanate From a Diamine by Tosyl Chloride Method

A 250 mL round bottomed flask is charged with octadeca-1,18-diamine (7.9 grams, 27.7 mmol), triethylamine (20.0 mL, 144.8 mmol), and THF (120 mL), and then cooled with an ice bath under N₂ atmosphere. Carbon disulfide (1.70 mL, 28.3 mmol) is then added to the reaction mixture by syringe pump over 45 min. After the addition, the reaction mixture is then stirred at room temperature for 1 hour. The reaction process is monitored by ¹H NMR. The reaction mixture is cooled with an ice bath, and tosyl chloride (5.47 grams, 28.8 mmol) is added, and the reaction mixture is warmed to room temperature. After 30 minutes, 1 N HCl (30 mL) is added to the reaction mixture and allowed to form octadeca-1,18-diisothiocyanate and triethyl ammonium tosylate. Water is added to the reaction mixture and warmed to 60° C. to separate octadeca-1,18-diisothiocyanate, which floats to the surface. The octadeca-1,18-diisothiocyanate is filtered and solidified by cooling, washed with water, and dried.

### Example 7

### Preparation of a Diisothiocyanate From a Diamine by Water/Metal Nitrate Method

About 54 grams (43 cc, 0.71 mol) of carbon disulfide and 90 mL (1.3 mol) of concentrated aqueous ammonia are mixed in a round bottom flask. 170.7 grams (0.6 mol) of octadeca-1,18-diamine is added into the mixture at a slow rate such that the addition is complete in about 40 minutes. The stirring is continued for 30 minutes or until all the octadeca-1,18-diamine is dissolved, and the reaction mixture is allowed to stand for another 30 minutes to form ammonium dithiocarbamate salt.

The ammonium dithiocarbamate salt obtained above is dissolved in 800 mL of water and transferred to a 5 L round-bottomed flask. A solution of 400 grams (1.2 mol) of lead nitrate in 800 mL of water is added with constant stirring. Lead sulfide separates out as a heavy brown precipitate. The water is heated to 60° C. to melt the product octadeca-1,18-diisothiocyanate, which floats to the surface. The octadeca-1,18-diisothiocyanate is filtered and solidified by cooling, washed with water, and dried.

### Example 8

### Preparation of a Polythiourea Film

Octadeca-1,18-diamine (284.5 grams, 1000 mmol, 2000 mmol amine equivalents) is combined with octadeca-1,18-diisothiocyanate (368.6 grams, 1000 mmol, 2000 mmol isothiocyanate equivalents). The compounds are gently heated together to 60° C. with stirring to ensure uniform melt. The viscosity builds and the mixture solidifies after about 4 hours into a tough plastic-like film. The mixture is allowed to cure for about 12 hours. The film can be used as a packaging material.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (example, bodies of the appended claims) are generally intended as “open” terms (example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A method of preparing a polythiourea composition, the method comprising:
combining at least one polyamine compound, at least one polyisothiocyanate compound, and at least one catalyst to form a reaction mixture; and
mixing the reaction mixture to form the polythiourea composition;
wherein the at least one polyamine compound has an amine functionality of two or more, and is present in an amount of about 14 wt % to about 40 wt % of the reaction mixture;
wherein the at least one polyisothiocyanate compound has a functionality of two or more, and is present in an amount of about 60 wt % to about 86 wt % of the reaction mixture; and
wherein the at least one catalyst is present in an amount of about 0 wt % to about 5 wt % of the reaction mixture.

2. The method of claim 1, wherein the at least one polyamine compound comprises a primary amine, a secondary amine, a tertiary amine, or any combination thereof.

3. The method of claim 1, wherein the at least one polyamine compound comprises an aromatic polyamine, an aliphatic polyamine, a polyamino polyether, a polyamino polyester, a polyamino polycarbonate, a polyamino polycaprolactone, a polyamino acrylic, a polyamino styrenic, a polyamino polyamide, or any combination thereof.

4. The method of claim 1, wherein the at least one polyamine compound comprises an aromatic diamine, a C₅-C₂₅ alkyl diamine, a C₅-C₂₅ alkenyl diamine, a C₅-C₂₅ alkynyl diamine, or any combination thereof.

5. The method of claim 1, wherein the at least one polyisothiocyanate compound comprises an alkyl diisothiocyanate, an alkyl triisothiocyanate, an alkenyl diisothiocyanate, an alkenyl triisothiocyanate, an aryl diisothiocyanate, an aryl triisothiocyanate, or any combination thereof.

6. The method of claim 1, wherein the at least one polyisothiocyanate compound comprises a C₅-C₂₅ alkyl diisothiocyanate, a C₅-C₂₅ alkenyl diisothiocyanate, a C₅-C₂₅ alkynyl diisothiocyanate, or any combination thereof.

7. The method of claim 1, wherein the at least one polyamine compound is 2-{2-[2-(2-amino-ethoxy)-ethoxy]-ethoxy}-ethylamine and the at least one polyisothiocyanate compound is 4,4′-methylene diphenyl diisothiocyanate.

8. The method of claim 1, wherein the at least one polyamine component is octadeca-1,18 diamine and the at least one polyisothiocyanate component is octadeca-1,18 diisothiocyanate.

9. The method of claim 1, wherein the at least one catalyst comprises a bismuth catalyst, a zinc catalyst, a tin catalyst, an amine, or any combination thereof.

10. The method of claim 1, wherein the at least one catalyst comprises zinc octoate, stannous octoate, bis-butyltin dilaurate, bis-butyltin diacetate, bis-butyltin dimethoxide, dimethyl-bis[1-oxonedecyl)oxy]stannane, di-n-octyltin bis-isooctyl mercaptoacetate, triethylenediamine, triethylamine, 1,3,5-cycclohexyltriamine, tributylamine, N-ethylmorpholine, N-methyl-morpholine, N-methylpiperazine, trimethylene diazine, or any combination thereof.

11. The method of claim 1, wherein the polythiourea composition formed is a foam having a density of about 10 to about 250 kilograms per cubic meter.

12. The method of claim 1, wherein the polythiourea composition has a NCS content from about 2 wt % to about 80 wt % of the total polymer weight.

13. A method of making a polyisothiocyanate compound, the method comprising:
contacting a polyamine compound with carbon disulfide to form a thiocarbamate compound; and
contacting the thiocarbamate compound with an oxidizing agent to form the polyisothiocyanate compound.

14. The method of claim 13, wherein the polyamine compound comprises an aromatic polyamine, an aliphatic polyamine, a polyamino polyether, a polyamino polyester, a polyamino polycarbonate, a polyamino polycaprolactone, or any combinations thereof.

15. The method of claim 13, wherein the polyamine compound comprises an aromatic diamine, a C₅-C₂₅ alkyl diamine, a C₅-C₂₅ alkenyl diamine, a C₅-C₂₅ alkynyl diamine, or any combination thereof.

16. The method of claim 13, wherein the polyisothiocyanate compound comprises an alkyl diisothiocyanate, an alkyl triisothiocyanate, an alkenyl diisothiocyanate, an alkenyl triisothiocyanate, an aryl diisothiocyanate, an aryl triisothiocyanate, or any combination thereof.

17. The method of claim 13, wherein contacting the polyamine with carbon disulfide comprises contacting the polyamine with carbon disulfide in the presence of a base.

18. A method for preparing a chemical resistant coating composition, the method comprising:
contacting at least one polyamine component, at least one polyisothiocyanate component, and at least one catalyst to form a reaction mixture, and
mixing the reaction mixture to form a polythiourea prepolymer binder,
wherein the at least one polyamine component has an amine functionality of two or more, and is present in about 14 weight percent to about 40 weight percent of the reaction mixture,
the at least one polyisothiocyanate component has a functionality of two or more, and is present in about 60 weight percent to about 86 weight percent of the reaction mixture, and
the at least one catalyst is present in about 0 to about 5 weight percent of the reaction mixture.

19. The method of claim 18, wherein the at least one polyamine component comprises a primary amine, a secondary amine, a tertiary amine, or any combinations thereof.

20. The method of claim 18, wherein the at least one polyamine component comprises an aromatic polyamine, an aliphatic polyamine, a polyamino polyether, a polyamino polyester, a polyamino polycarbonate, a polyamino polycaprolactone, or any combinations thereof.

21. The method of claim 18, wherein the at least one polyisothiocyanate component comprises an alkyl diisothiocyanate, an alkyl triisothiocyanate, an alkenyl diisothiocyanate, an alkenyl triisothiocyanate, an aryl diisothiocyanate, an aryl triisothiocyanate, or any combination thereof.

22. The method of claim 18, wherein the at least one catalyst comprises a bismuth catalyst, a zinc catalyst, a tin catalyst, an amine, or any combination thereof.

23. The method of claim 18, wherein the coating further comprises a solvent, a pigment, a coalescing agent, a rheology modifier, a plasticizer, or any combination thereof.

24. The method of claim 18, wherein the chemical resistant coating is a decorative coating, an industrial coating, a protective coating, a UV-protective coating, a self-cleaning coating, or any combination thereof.
